(19) United States
(12) Patent Application Publication
(10) Pub. No.: US 2003/0031628 A1
(43) Pub. Date: Feb. 13, 2003

(54) Imaging infection using fluorescent protein as a marker

(76) Inventors: Ming Zhao, San Diego, CA (US); Meng Yang, San Diego, CA (US); Mingxu Xu, La Jolla, CA (US)

Correspondence Address: Kate H. Murashige, Morrison & Foerster LLP, Suite 500, 3811 Valley Centre Drive, San Diego, CA, 92130, US

(21) Appl. No.: 10/192,740

(22) Filed: Jul. 9, 2002

Related U.S. Application Data
(60) Provisional application No. 60/304,223, filed on Jul. 9, 2001.

(51) Int. Cl. A61K 49/00

(52) U.S. Cl. 424/9.6

(57) ABSTRACT

A method to follow the progress of infection in vertebrate subjects utilizes infective agents which have been modified to express a fluorescent protein. The method can also monitor expression of genes associated with infective agents during the course of infection. The method may further include targeting tumors with the modified infective agents.

### TECHNICAL FIELD

The invention relates to the study of microbial and viral infection. Specifically, it concerns systems for studying progress of, and control of, infection in vertebrates and methods for evaluating candidate drugs and targeting tumors.

### BACKGROUND ART

The use of green fluorescent protein to visualize cancer progression and metastasis is by now well established. See, for example, Hoffman, R. M., Methods in Enzymology (1999) 302:20-31 (P. Michael Conn, ed., Academic Press, San Diego). The use of whole body imaging to chart real time progression and to assess the efficacy of proposed protocols for treating tumors is disclosed in U.S. Pat. No. 6,251,384, the contents of which are incorporated herein by reference.

The advantages of green fluorescent protein have been noted in that it does not require any substrates or cofactors and its expression in living cells does not apparently cause any biological damage. In addition, the fluorescence emitted makes this a particularly sensitive technique. Indeed, the whole body images obtainable using simple equipment, e.g., 490 nm excitation from a xenon or mercury lamp along with image capture by a CCD color video camera permit real-time investigations of tumor growth and metastasis. See, for example, Yang, M., et al., Proc. Natl. Acad. Sci. USA (2000) 97:1206-1211.

The present invention extends the techniques developed in imaging tumor growth and metastasis to the study of infection. Microbial and viral infection can be monitored by labeling the infectious agent with a bright fluorescent protein and the progress of infection monitored. In addition, protocols useful in treating microbial or viral infection can be evaluated by taking advantage of this technique. The materials and methods for obtaining suitable expression of fluorescent proteins are readily available. For example, Cheng, L., et al., Gene Therapy (1997) 4:1013-1022, describe the modification of hematopoietic stem cells with green fluorescent protein (GFP) encoding sequences under control of a retroviral promoter. Although the authors state that human stem cells are transfected with this system only with difficulty, by using an enhanced form of the GFP, satisfactory brightness could be achieved. Grignani, F., et al., Cancer Res (1998) 58:14-19, report the use of a hybrid EBV/retroviral vector expressing GFP to effect high-efficiency gene transfer into human hematopoietic progenitor cells.

Vectors containing various modified forms of GFP to provide various colors are marketed by Clontech. The Clontech vectors intended for mammalian cell expression place the GFP under control of the cytomegalovirus (CMV) promoter; such expression systems can also be used to label viral infectious agents.

Attempts have been made to visualize bacteria in mammalian subjects using luciferase as a marker, but because of the low luminosity of this system, whole body imaging is not practical. See, for example, Contag, P. R., et al., Nat. Med. (1998) 4:245-247.

GFP expressing bacteria have been previously employed in a number of studies that were not in intact, living animals (Wu, H., et al., Microbiol. (2000) 146:2481-2493; Ling, S. H. M., et al., Microbiol. (2000) 146:7-19; Badger, J. L., et al., Mol. Microbiol. (2000) 36(1):174-182; Kohler, R., et al., Mol Gen. Genet. (2000) 262:1060-1069; Valdivia, R. H., et al., Gene (1996) 173:47-52; Valdivia, R. H., et al., Science (1997) 277:2007-2011; Scott, K. P., et al., FEMS Microbiol Ltrs. (2000) 182:23-27; Prachaiyo, P., et al., J. Food Protect. (2000) 63:427-433; Geoffroy, M-C., Applied & Env. Microbiol. (2000) 66:383-391). An example of such studies was the visualization of the in vitro infection of muscle tissue by the pathogenic E. coli O157H GFP (Prachaiyo, P., et al., supra). Another approach examined the mouse gastrointestinal tract after gavage infection by removal and fixation of the gastrointestinal tissue (Geoffroy, M-C., supra). Fish infected with GFP transduced Edwardsiella tarda were imaged for infection after removal of their organs (Ling, S. H. M., et al., supra). Genes associated with virulence and other infectious processes were evaluated by linkage to GFP expression (Ling, S. H. M., et al., supra; Badger, J. L., et al., supra; Kohler, R., et al., supra; Valdivia, R. H., et al., supra (1996).

The present invention also extends to targeting tumors to deliver therapeutics thereto via infective agents such as microorganisms using fluorescence. Attempts have been made to deliver the anaerobic bacteria Clostridia novyi to necrotic regions in tumors (Dang, L. H., et al., Proc. Natl. Acad. Sci. USA (2001) 98:15155-15160). In addition, the necrotic regions of tumors have been targeted using Bifidobacterium longum (Yazawa, K., et al., Cancer Gene Therapy (2000) 7(2):269-274 and Yazawa, K., et al., Breast Cancer Res. & Treatment (2001) 66:165-170). These approaches depend on anarobes, are targeted at necrotic tissue only and/or may be used only for tumors of a large size. Further, tumors have been targeted using Salmonella that is devoid of its toxin (Low, K. B., et al., Nature Biotech. (1999) 17:37-41). Additional studies have reported the tumor targeting capability of Salmonella in human patients with metastatic melanoma and renal cell carcinoma (Toso, J. F., et al., J. Clin. Oncol. (2002) 20(1):142-152). These approaches do not provide a way to visualize the bacteria in living animals.

Bacteria and other microorganisms offer many features to deliver therapeutics to tumors. For example they are readily transformed to produce both human and specialized bacterial proteins. The bacterial proteins, however, include a wide variety and potency of toxins. In order to take advantage of such powerful molecules, it would be useful to have an accurate tumor-targeting mechanism for therapeutic-delivering bacteria as shown by the present invention.

### DISCLOSURE OF THE INVENTION

The invention provides models which permit the intimate study of formation of microbial or viral infection in a realistic and real-time setting. By using fluorescent proteins such as green fluorescent protein (GFP) as a stable and readily visualized marker, the progression of infection can be modeled and the mechanism elucidated. The invention is also directed, in part, to tumor targeting which depends on the ability to visualize the bacteria or microorganism as well as its therapeutic molecule.

Thus, in one aspect, the invention is directed to a method to monitor the course of infection in a model vertebrate system by monitoring the spatial and temporal progression of fluorescence in said vertebrate subject wherein said subject has been subjected to infection by a microbe or virus which microbe or virus expresses a fluorescent protein.

In another aspect, the invention is directed to a method to evaluate a candidate protocol or drug for inhibition of infection in a subject which method comprises administering the protocol or drug to a vertebrate subject which has been infected with a microbe or virus that expresses a fluorescent protein and monitoring the temporal and spatial progress of infection by observing the presence, absence or intensity of fluorescence at various locations at various times in the infected subject. In this method, in addition, the presence, absence or intensity of fluorescence at various locations in a control subject at various times is also monitored for comparison with the subject that has been treated with the protocol or drug. The progress of infection over time and space is compared in the treated subject and the control subject, and a diminution of the intensity of infection in the treated subject as compared to the control subject identifies a successful protocol or drug.

In yet another aspect, the invention is directed to a method to target tumors using a therapeutic infective agent in a vertebrate subject comprising administering an infective agent that expresses a fluorescent protein to the vertebrate subject and observing the presence, absence, or intensity of fluorescence at various locations in the subject as a function of time. Preferably the therapeutic infective agent targets the tumor and delivers a therapeutic product to the tumor.

The methods of the invention can also be used to monitor the nature of the microbial or viral systems that are significant in the progress of infection by coupling the nucleotide sequence encoding the fluorescent protein to various positions in the genome of the microbe or virus and monitoring the expression of the fluorescent protein by monitoring the fluorescence.

Lastly, the invention is directed to a tumor-targeting infective agent that expresses a fluorescent protein that is capable of targeting tumors in intact, living mammals in comparison to normal cells.

### BRIEF DESCRIPTION OF THE DRAWINGS

FIGS. 1A-1H show the locations of fluorescence in various parts of a mouse administered 10¹¹ E. coli-GFP by gavage. FIG. 1A shows evidence of infection in the stomach immediately after gavage; FIGS. 1B-1G show the presence of fluorescence in the small intestine 10, 20, 30, 40, 50 and 60 minutes after gavage, respectively. FIG. 1H shows the presence of infection in the colon 120 minutes after gavage.

FIGS. 2A-2C show the results of intravital imaging of E. coli after gavage with 10¹¹ E. coli-GFP. As shown in FIG. 2A, GFP infection is present in the stomach and the duodenum immediately after gavage; FIG. 2B shows the presence of infection in the small intestine 40 minutes after gavage; FIG. 2C shows the presence of infection in the colon 120 minutes after gavage.

FIGS. 3A-3B show whole body and intravital imaging of infection in the stomach, small intestine and colon after gavage. FIG. 3A shows a whole body image in the stomach (arrowhead), small intestine (fine arrows), and colon (thick arrow) after multiple gavage of aliquots of 3×10¹¹ E. coli-GFP. FIG. 3B shows corresponding intraviral images labeled similarly.

FIG. 4 shows the results of whole body imaging of infection in the colon immediately after enema of 10¹¹ E. coli-GFP.

FIGS. 5A-5D show the results of whole body imaging of peritoneal cavity infection in antibiotic response. FIGS. 5A and 5C show the infection in the peritoneal cavity immediately after intraperitoneal (i.p.) injection of 10⁹ E. coli-GFP. FIG. 5B shows an untreated mouse six hours after injection; the animal died at six hours. FIG. 5D shows a Kanamycin treated mouse six hours after i.p. injection, wherein the animal survived.

FIG. 6 shows the results of intravital imaging of intraperitoneal infection as described in FIG. 5.

FIG. 7A shows whole body imaging of an RFP-labeled U-87 human glioma growing in a nude mouse. FIG. 7B shows fluorescence-guided injection of a PBS solution containing GFP-labeled Salmonella. FIG. 7C shows whole body imaging of a GFP-labeled Salmonella in the RFP-labeled U-87 human glioma immediately after injection. FIG. 7D shows the GFP-labeled Salmonella growing in the RFP-labeled U-87 human glioma one day after injection.

FIG. 8A shows whole body imaging of an RFP-labeled DU-145 human prostate tumor in a nude mouse (Mouse 1). FIG. 8B shows GFP-labeled Salmonella injected in the tumor of Mouse 1 imaged immediately after injection. FIG. 8C shows whole body imaging of an RFP-labeled DU-145 human prostate tumor in a nude mouse (Mouse 2). FIG. 8D shows the results of GFP-labeled Salmonella injected in the RFP-labeled DU-145 human prostate tumor which was imaged immediately after injection in Mouse 2.

FIG. 9A shows whole body imaging of an RFP-labeled MDA MB-435 human breast tumor growing in a nude mouse. FIG. 9B shows whole body imaging of GFP-labeled Salmonella injected in the tumor immediately after injection.

FIG. 10A shows whole body imaging of an RFP-labeled U-87 human glioma growing in a nude mouse. FIG. 10B shows whole body imaging of a PBS solution containing GFP-labeled Salmonella injected in the glioma. FIG. 10C shows whole body imaging of a GFP-labeled Salmonella in the RFP-labeled U-87 human glioma immediately after injection. FIG. 10D shows whole body imaging of a GFP-labeled Salmonella growing in the RFP-labeled U-87 human glioma one day after injection.

FIG. 11A shows whole body imaging of an RFP-labeled DU-145 human prostate tumor in a nude mouse (Mouse 1). FIG. 11B shows the results of GFP-labeled Salmonella injected in the RFP-labeled DU-145 human prostate tumor which was imaged immediately after injection in Mouse 1. FIG. 11C shows whole body imaging of an RFP-labeled DU-145 human prostate tumor in a nude mouse (Mouse 2). FIG. 11D shows the results of GFP-labeled Salmonella injected in the RFP-labeled DU-145 human prostate tumor which was imaged immediately after injection in Mouse 2.

FIG. 12A shows whole body imaging of an RFP-labeled MDA MB-435 human breast tumor growing in a nude mouse. FIG. 12B shows the results of GFP-labeled Salmonella injected in the tumor which was imaged immediately after injection.

FIG. 13A shows whole body imaging of a GFP-labeled PC-3 human prostate tumor growing in a nude mouse. FIG. 13B shows the results of RFP-labeled Salmonella injected in the tumor which was imaged immediately after injection. FIG. 13C shows whole body imaging of an RFP-labeled Salmonella growing in the GFP-labeled PC-3 human prostate tumor one day after injection.

FIG. 14A shows whole body imaging of a GFP-labeled PC-3 human prostate tumor growing in a nude mouse. FIG. 14B shows the results of RFP-labeled Salmonella injected in the GFP-labeled PC-3 human prostate tumor immediately after injection. FIG. 14C shows whole body imaging of an RFP-labeled Salmonella growing in the GFP-labeled PC-3 human prostate tumor one day after injection. FIG. 14D shows whole body imaging of an RFP-labeled Salmonella growing in the GFP-labeled PC-3 human prostate tumor four days after injection.

FIG. 15A shows whole body imaging of a GFP-labeled PC-3 human prostate tumor growing in a nude mouse. FIG. 15B shows the results of RFP-labeled Salmonella injected in the GFP-labeled PC-3 human prostate tumor which was imaged immediately after injection. FIG. 15C shows whole body imaging of an RFP-labeled Salmonella growing in the GFP-labeled PC-3 human prostate tumor one day after injection. FIG. 15D shows whole body imaging of an RFP-labeled Salmonella growing in the GFP-labeled PC-3 human prostate tumor four days after injection.

FIG. 16 shows RFP-labeled Salmonella targeting and progressively growing in GFP-labeled PC-3 human prostate tumor growing in nude mice demonstrated by histology. RFP-labeled Salmonella growing in the GFP-labeled PC-3 human prostate tumor four days after injection (FIG. 15D).

FIGS. 17A-17B shows the effect of treatment of RFP-labeled Salmonella on PC-3 human prostate tumor growing in nude mice demonstrated by histology. FIG. 17A is the untreated control. FIG. 17B is the treatment after RFP-labeled Salmonella.

### MODES OF CARRYING OUT THE INVENTION

The invention provides model systems for the study of the mechanism of infection. Advantage is taken of visible marker fluorescence proteins to label the infectious agents so that their migration and colonization in tissues can be followed as the infection progresses.

As used herein, “progression of infection” refers to the general time-dependent manner in which infective agent and infected cells migrate and/or proliferate through an infected organism. The progress of infection may be a function simply of the location of the infectious agent or infected cells but generally also is a function of the proliferation of the infective agent and infected cells. Thus, both the location and intensity of fluorescence are significant in monitoring progression.

Since sufficient intensity can be achieved to observe the migration of fluorescent cells in the intact animal, in addition to determining the migration of the infectious agent by excising organs or tissue, if desired, the progression of metastasis can be observed in the intact subject. Either or both methods may be employed to observe the progress of infection and in evaluating, in model systems, the efficacy of potential protocols and drugs.

In addition, the present invention takes advantage of delivering therapeutics by infective agents to tumors and provides an accurate tumor targeting mechanism. It is advantageous in tumor targeting to be able to visualize the infective agent as well as its therapeutic molecule. Some advantages of fluorescence guided injection of tumors are that there is no lower limit to the size of tumor that can be treated, and further, the method is independent of tumor necrosis. In addition, infective agents are not limited to anarobes nor non-virulent strains of infective agents. A “therapeutic,” “therapeutic molecule” or “therapeutic product” as used herein refers to a gene of interest that is contained in an infective agent, or a product secreted from the infective agent, such as a toxin or other therapeutic protein, or a product that is not secreted but which is used by the infective agent such that a therapeutic effect on tumor is affected. A gene of interest means any gene that has a therapeutic effect on tumor such as a gene that expresses an anti-tumor agent. Examples of a therapeutic molecule is a gene expressing methioninase or methioninase itself as disclosed in U.S. Pat. No. 6,231,854. Other examples include p53, BAX, toxins, tumor necrosis factor (TNF), TNF-related apoptosis-inducing ligand, Fas ligand, and antibodies against death receptors.

The label used in the various aspects of the invention is a fluorescent protein. The native gene encoding the seminal protein in this class, green fluorescent protein (GFP) has been cloned from the bioluminescent jellyfish Aequorea victoria (Morin, J., et al., J. Cell Physiol (1972) 77:313-318). The availability of the gene has made it possible to use GFP as a marker for gene expression. The original GFP itself is a 283 amino acid protein with a molecular weight of 27 kD. It requires no additional proteins from its native source nor does it require substrates or cofactors available only in its native source in order to fluoresce. (Prasher, D.C., et-al., Gene (1992) 111:229-233; Yang, F., et al., Nature Biotechnol (1996) 14:1252-1256; Cody, C. W., et al., Biochemistry (1993) 32:1212-1218.) Mutants of the original GFP gene have been found useful to enhance expression and to modify excitation and fluorescence, so that “GFP” in various colors, including reds and blues has been obtained. GFP-S65T (wherein serine at 65 is replaced with threonine) is particularly useful in the present invention method and has a single excitation peak at 490 nm. (Heim, R., et al., Nature (1995) 373:663-664); U.S. Pat. No. 5,625,048. Other mutants have also been disclosed by Delagrade, S., et al., Biotechnology (1995) 13:151-154; Cormack, B., et al., Gene (1996) 173:33-38 and Cramer, A., et al., Nature Biotechnol (1996) 14:315-319. Additional mutants are also disclosed in U.S. Pat. No. 5,625,048. By suitable modification, the spectrum of light emitted by the GFP can be altered. Thus, although the term “GFP” is often used in the present application, the proteins included within this definition are not necessarily green in appearance. Various forms of GFP exhibit colors other than green and these, too, are included within the definition of “GFP” and are useful in the methods and materials of the invention. In addition, it is noted that green fluorescent proteins falling within the definition of “GFP” herein have been isolated from other organisms, such as the sea pansy, Renilla reniformis. Any suitable and convenient form of GFP can be used to modify the infectious agents useful in the invention, both native and mutated forms.

In order to avoid confusion, the simple term “fluorescent protein” will be used; in general, this is understood to refer to the fluorescent proteins which are produced by various organisms, such as Renilla and Aequorea as well as modified forms of these native fluorescent proteins which may fluoresce in various visible colors, such as red, yellow, and cobalt, which are exhibited by red fluorescent protein (RFP), yellow fluorescent protein (YFP) or cobalt fluorescent protein (CFP), respectively. In general, the terms “fluorescent protein” and “GFP” or “RFP” are used interchangeably.

Because fluorescent proteins are available in a variety of colors, imaging with respect to more than a single color can be done simultaneously. For example, two different infective agents or three different infective agents each expressing a characteristic fluorescence can be administered to the organism and differential effects of proposed treatments evaluated. In addition, a single infectious organism could be labeled constitutively with a single color and a different color used to produce a fusion with a gene product either intracellular or that is secreted. Thus, the nucleotide sequence encoding a fluorescent protein having a color different from that used to label the organism per se can be inserted at a locus to be studied or as a fusion protein in a vector with a protein to be studied. As a further illustration, toxins and other potentially therapeutic proteins will be genetically linked with RFP in order to label and visualize the therapeutic product of GFP-labeled bacteria and visa versa. Two-color imaging will be used to visualize targeting of the bacteria to the tumor as well as their secreted therapeutic product. These tumor-targeting bacteria will be adapted for selective growth in tumors as visualized by their fluorescence. Further, one or more infective agents could each be labeled with a single color, a gene of interest with another color, and the tumor with a third color. For example, fluorescence-expressing tumors in laboratory animals will enable visualization of tumor targeting of fluorescence-labeled infective agents by whole body imaging, as well as the infective agents' therapeutic product.

As exemplified herein, GFP- and RFP-labeled bacteria were delivered by fluorescence-guided injection in GFP- and RFP-labeled tumors implanted in nude mice and thus the bacteria was targeted to GFP-labeled tumor, thereby inducing tumor necrosis. In particular, the targeting of GFP-and RFP-labeled E. coli and S. typhimurium to RFP- and GFP-expressing tumors in mice was visualized by dual-color whole-body imaging. GFP- and RFP-labeled bacteria growing in targeted RFP- and GFP-labeled tumors have been visualized by dual-color whole-body imaging as shown in the Examples herein. Thus, tumor targeting of fluorescent labeled microorganisms has been shown. The method of the invention can also be used, however, to monitor the mis-targeting of the infective agent in order ultimately to select for bacteria that targets tumors.

Techniques for labeling cells in general using GFP are disclosed in U.S. Pat. No. 5,491,084 (supra).

The methods of the invention utilize infectious agents which have been modified to express the nucleotide sequence encoding a fluorescent protein, preferably of sufficient fluorescence intensity that the fluorescence can be seen in the subject without the necessity of any invasive technique. While whole body imaging is preferred because of the possibility of real-time observation, endoscopic techniques, for example, can also be employed or, if desired, tissues or organs excised for direct or histochemical observation.

The nucleotide sequence encoding the fluorescent protein may be introduced into the infectious agent by direct modification, such as modification of a viral genome to locate the fluorescent protein encoding sequence in a suitable position under the control sequences endogenous to the virus, or may be introduced into microbial systems using appropriate expression vectors. Infective agents may be bacteria, eukaryotes such as yeast, protozoans such as malaria, or viruses. A multiplicity of expression vectors for particular types of bacterial, protozoan, and eukaryotic microbial systems is well known in the art. A litany of control sequences operable in these systems is by this time well understood. The infectious agent is thus initially modified either to express the fluorescent protein under control of a constitutive promoter as a constant feature of cell growth and reproduction, or may be placed in the microbial or viral genome at particular desired locations, replacing endogenous sequences which may be involved in virulence or otherwise in the progress of infection to study the temporal and spatial parameters characteristic of expression of these endogenous genes. Thus, it is possible to explore the types of factors endogenous to the microbe or virus which contribute to the effectiveness of the infection by suitable choice of positioning. Similarly, a gene expressing a fluorescent protein may be introduced into tumor cells such that laboratory animals contain tumors that can be visualized. Another approach to prepare fluorescent tumors is through photo dynamic therapy (PDT) where the tumor absorbs agents that fluoresce such as clinically approved agents, for example, hematoporphorins

The appropriately modified infectious agent is then administered to the subject in a manner which mimics, if desired, the route of infection believed used by the agent or by an arbitrary route. Administration may be by injection, gavage, oral, by aerosol into the respiratory system, by suppository, by contact with a mucosal surface in general, or by any suitable means known in the art to introduce infectious agents. In tumor targeting where the tumor expresses a fluorescent protein, administration can be made by fluorescent guided injection. Unlike the situation with regard to the study of tumor metastasis using fluorescence, it is not necessary that the subject be immunocompromised since infection occurs readily in organisms with intact immune systems. However, immunocompromised subjects may also be useful in studying the progress of the condition.

Although endoscopy can be used as well as excision of individual tissues, it is particularly convenient to visualize the migration of infective agent and infected cells in the intact animal through fluorescent optical tumor imaging (FOTI). This permits real-time observation and monitoring of progression of infection on a continuous basis, in particular, in model systems, in evaluation of potential anti-infective drugs and protocols. Thus, the inhibition of infection observed directly in test animals administered a candidate drug or protocol in comparison to controls which have not been administered the drug or protocol indicates the efficacy of the candidate and its potential as a treatment. In subjects being treated for infection, the availability of FOTI permits those devising treatment protocols to be informed on a continuous basis of the advisability of modifying or not modifying the protocol. In one embodiment, to ascertain the feasibility of fluorescently-labeled bacteria to target tumors, GFP-labeled bacteria were injected into the Lewis lung tumor growing in nude mice. The tumor area became highly fluorescent and readily visualized by blue light excitation in a light box with a CCD camera and a GFP filter.

Suitable vertebrate subjects for use as models are preferably mammalian subjects, most preferably convenient laboratory animals such as rabbits, rats, mice, and the like. For closer analogy to human subjects, primates could also be used. Any appropriate vertebrate subject can be used, the choice being dictated mainly by convenience and similarity to the system of ultimate interest. Ultimately, the vertebrate subjects can be humans.

In is expected that tumor-targeting bacteria can be adapted for selective growth in tumors as vectors for tumor-selective gene therapy.

The following examples are intended to illustrate but not to limit the invention.

### Preparation A

### Modification of Infectious Agents

A variant of the Renilla mulleri green fluorescent protein (RMV-GFP) (Zhao, M., Xu, M., Hoffman, R. M., unpublished data) was cloned into the BamHI and NotI sites of the pUC19 derivative pPD16.38 (Clontech, Palo Alto, Calif.) with GFP expressed from the lac promoter. The vector was termed pRMV-GFP. pRMV-GFP was transfected into E. coli JM 109 competent cells (Stratagene, San Diego, Calif.) by standard methods, and transformed cells were selected by ampicillin resistance on agar plates. High expression E. coli-GFP clones were selected by fluorescence microscopy.

E. coli has also been labeled with RFP and, in addition, Salmonella typhimurium has been labeled with both the GFP and RFP.

### EXAMPLE 1

### Infection of Mice by Gavage

Nu/nu/CD-1 mice, 4 weeks old, female, mice were gavaged with 0.5 ml of an E. coli-GFP suspension (5×10¹⁰/ml) with a 20 gauge barrel tip feeding needle (Fine Science Tools Inc., Foster City, Calif.) and latex-free syringe (Becton Dickinson, Franklin Lakes, N.J.).

After gavage, at various time points, imaging of the mice was performed. Imaging was carried out in a light box illuminated by blue light fiber optics (Lightools Research, Inc., Encinitas, Calif.). Images were captured using a Hamamatsu C5810 3-chip cooled color CCD camera (Hamamatsu Photonics Systems, Bridgewater, N.J.). Images of 1024×724 pixels were captured directly on an IBM PC or continuously through video output on a high resolution Sony VCR model SLV-R1000 (Sony Corp., Tokyo, Japan). Images were processed for contrast and brightness and analyzed with the use of Image Pro Plus 3.1 software (Media Cybernetics, Silver Springs, Md.).

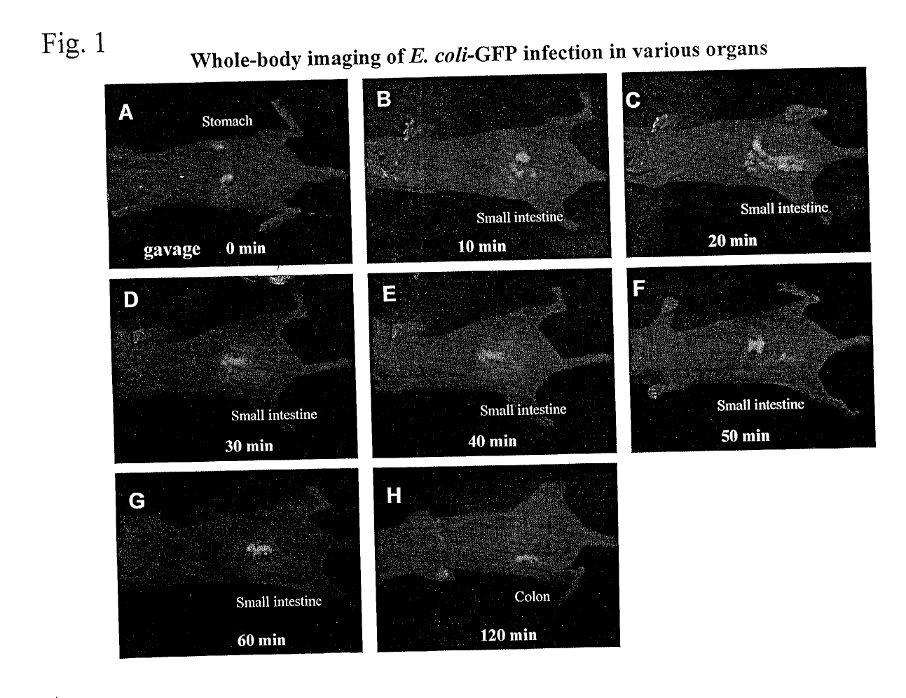

E. coli-GFP introduced to the mouse GI tract by gavage became visible in the stomach in whole body images almost immediately (FIG. 1A). The stomach emptied within 10 minutes post gavage and the E. coli-GFP next appeared in the small intestine (FIGS. 1B-G). The bacterial population in the small intestine appeared to peak at 40 minutes post gavage (FIG. 1E) and disappeared by 120 minutes (FIG. 1G). After 120 minutes, E. coli-GFP appeared in the colon (FIG. 1H).

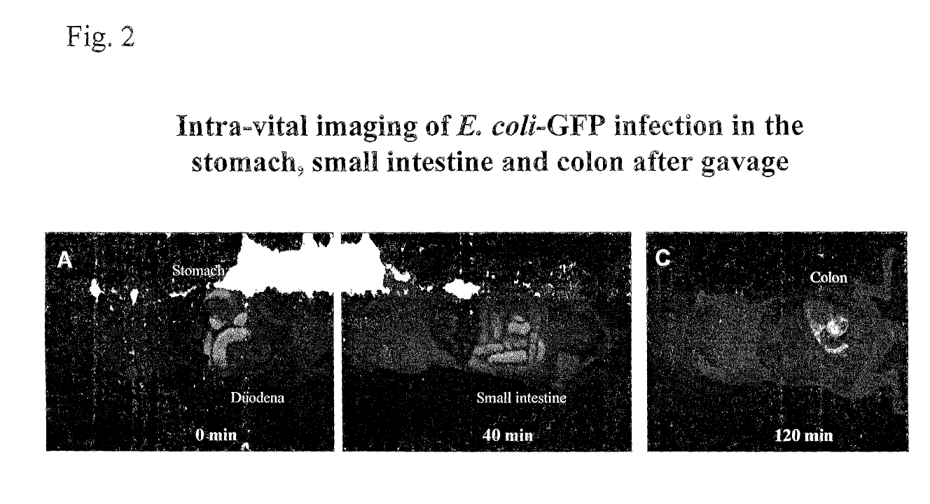

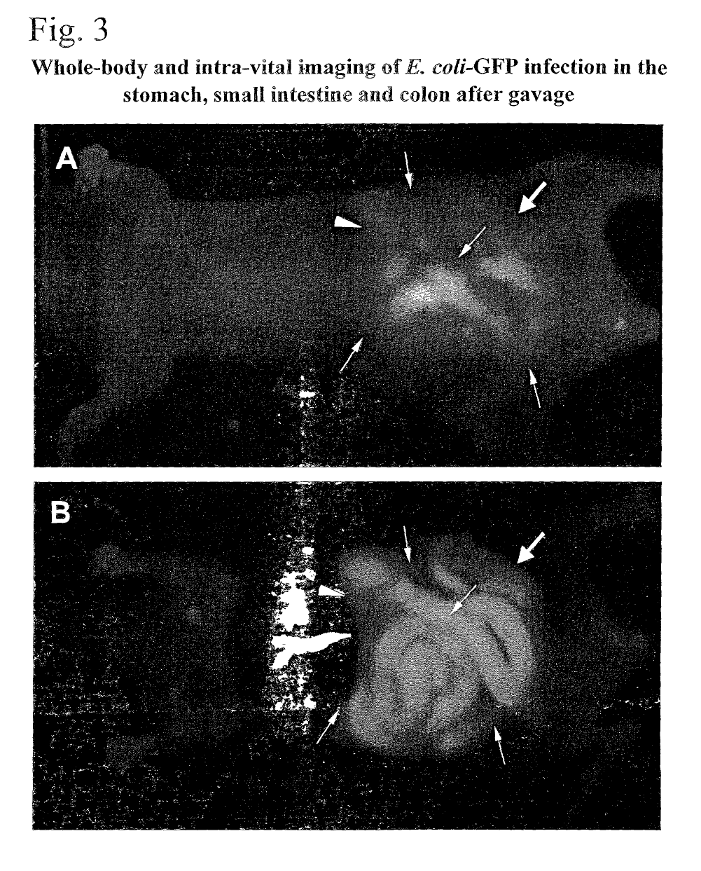

At appropriate times after gavage, the abdominal cavity was opened and intravital images made of the E. coli-GFP fluorescence. The stomach (FIG. 2A), small intestine (FIG. 2B), and colon (FIG. 2C) were brightly fluorescent with E. coli-GFP as seen by intravital imaging. Multiple gavage with E. coli-GFP allowed simultaneous inoculation of the stomach, small intestine, and colon, which were imaged by whole-body (FIG. 3A) and intravital techniques (FIG. 3B). Comparison of whole-body and intravital images of E. coli-GFP in the stomach, small intestine, and colon showed a high degree of correspondence.

### EXAMPLE 2

### E. coli-GFP Direct Colon Infection

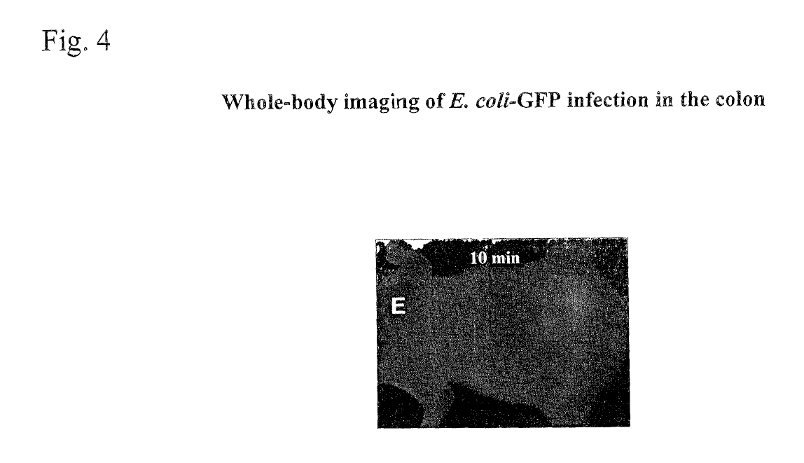

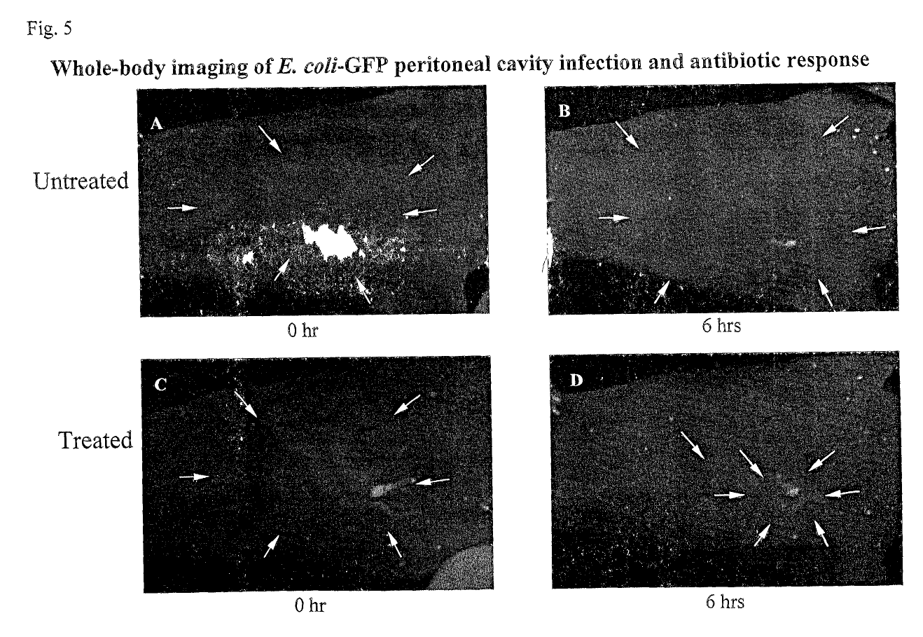

One and one half ml containing 3×10¹⁰ E. coli-GFP per mouse were administered into the colon by enema using a 20 gauge barrel tip feeding needle (Fine Science Tools Inc., Foster City, Calif.) and latex-free syringe (Becton Dickinson). These mice were also subjected to imaging using the techniques of Example 1. The results are shown in FIG. 4.

### EXAMPLE 3

### E. coli-GFP Peritoneal Infection and Response to Antibiotics

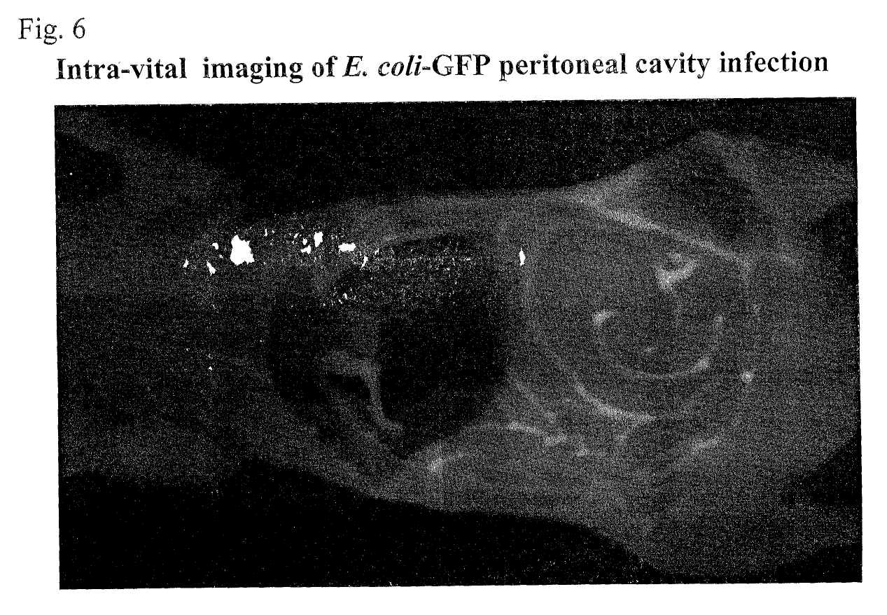

The mice in each group were given an intraperitoneal (i.p.) injection of 10⁹-10¹⁰ E. coli-GFP using a 1 ml 29G1 latex-free syringe (Becton Dickinson). Immediately after injection, the fluorescent bacteria were seen localized around the injection site by external whole-body imaging. (FIGS. 5A, C). Six hours later, the E. coli-GFP were seen to spread throughout the peritoneum (FIG. 5B), coinciding with the death of the animal. Intravital imaging of E. coli-GFP in the open peritoneal cavity at 6 hours (FIG. 6) showed a bacterial distribution similar to that seen by external whole-body imaging.

Another group of intraperitoneally infected animals were treated with 2 mg Kanamycin in 100 μl following inoculation. A control group of infected mice were given an i.p. injection of 100 μl of PBS instead of antibiotic. Whole-body imaging of treated mice showed a marked reduction of the bacterial population over the next six hours (FIGS. 5C, D).

### EXAMPLE 4

### Targeting in Brain Cancer Using Whole-Body Imaging

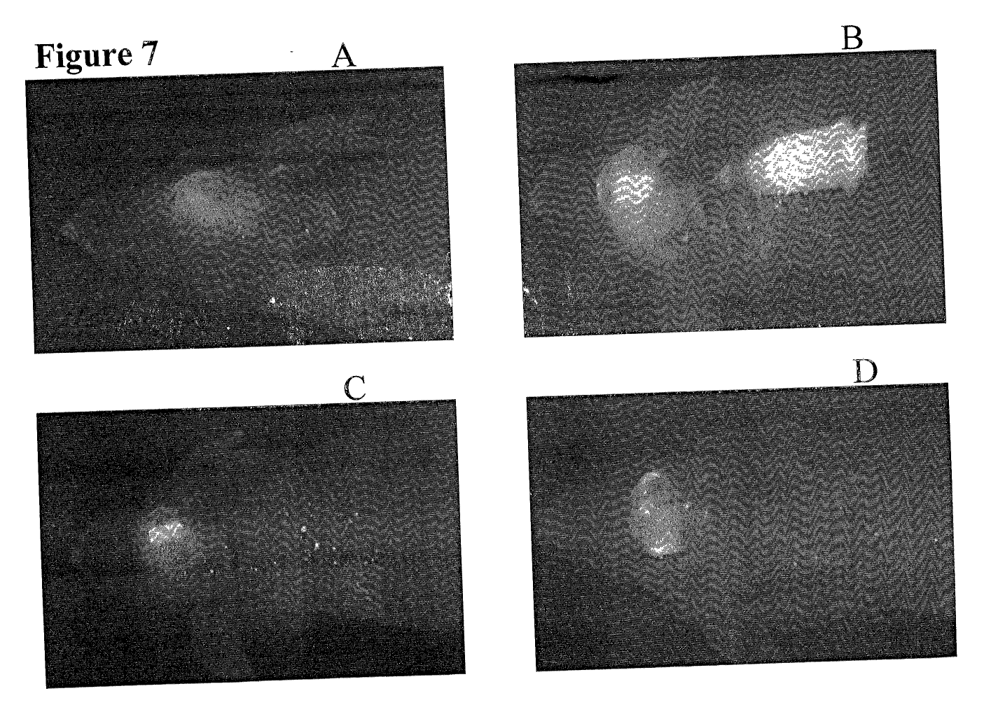

A PBS solution (10 μl) containing 1×10⁸ GFP-labeled Salmonella typhimurium was injected in the RFP-labeled U-87 human glioma in a nude mouse (FIG. 7A) using fluorescence guided injection (FIG. 7B). GFP-labeled Salmonella in the RFP-labeled U-87 human glioma was imaged using techniques similar to Example 1 immediately after injection (FIG. 7C). GFP-labeled Salmonella growing in the RFP-labeled U-87 human glioma one day after injection was seen showing GFP-labeled Salmonella localization around the tumor as well as reduction of tumor size (FIG. 7D).

### EXAMPLE 5

### Targeting in Prostate Tumors Using Whole-Body Imaging

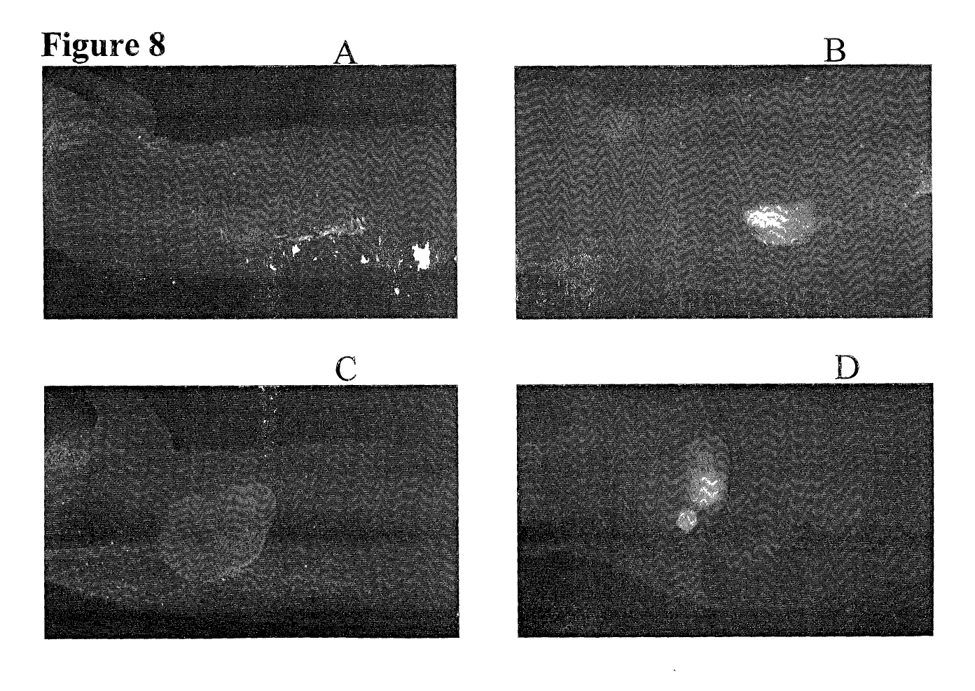

In a first nude mouse having an RFP-labeled DU-145 human prostate tumor (FIG. 8A), 1×10⁸ GFP-labeled Salmonella typhimurium was injected in the RFP-labeled DU-145 human prostate tumor and imaged using whole-body imaging immediately after injection (FIG. 8B). GFP-labeled Salmonella localization around the tumor (FIG. 8B) was seen. One advantage to fluorescent guided injection is that virulent Salmonella can be used and tumors of all sizes can be targeted.

In a second nude mouse having an RFP-labeled DU-145 human prostate tumor (FIG. 8C), a solution containing 2×10⁸ GFP-labeled Salmonella typhimurium was injected in the RFP-labeled DU-145 human prostate tumor and imaged using whole-body imaging immediately after injection. GFP-labeled Salmonella typhimurium localization in the tumor (FIG. 8D) was seen.

### EXAMPLE 6

### Targeting in Breast Cancer Using Whole-Body Imaging

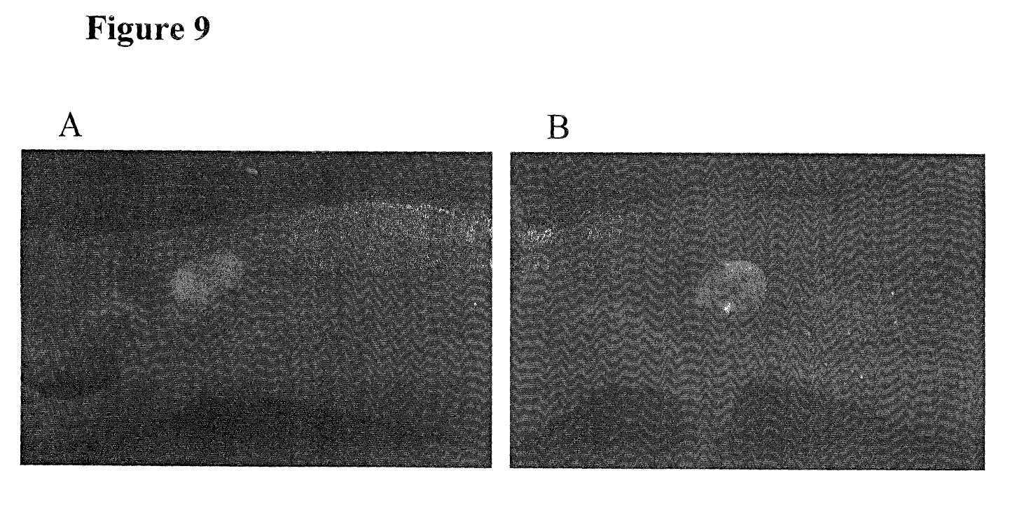

A solution containing 2×10⁸ GFP-labeled Salmonella typhimurium was injected in the RFP-labeled MDA MB-435 human breast tumor growing in a nude mouse (FIG. 9A) and imaged using techniques similar to Example 1 immediately after injection showing localization around the tumor (FIG. 9B) and apparent reduction of tumor size, indicating tumor necrosis.

### EXAMPLE 7

### Targeting in Prostate Tumor Using Whole-Body Imaging

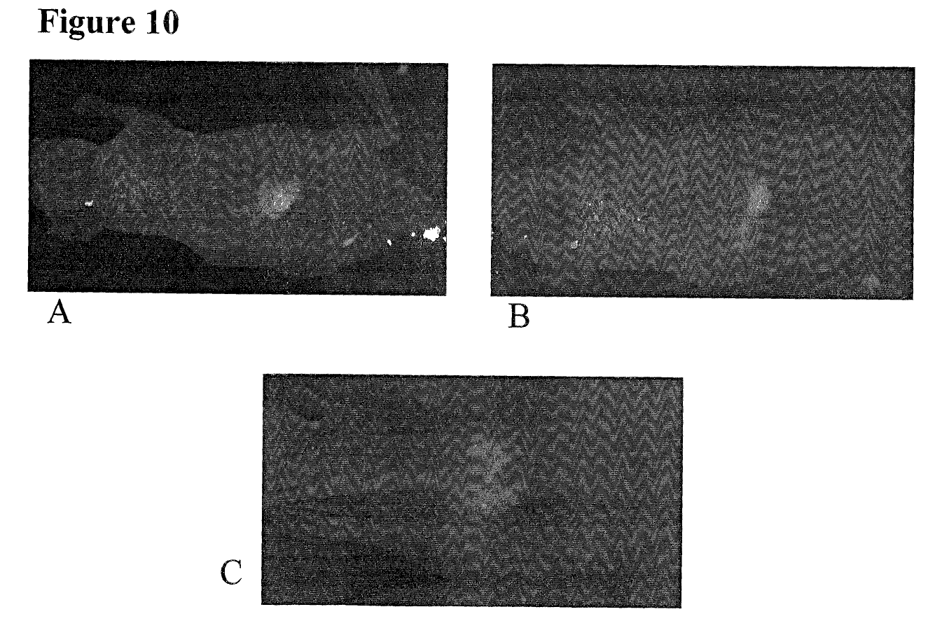

A solution containing 3×10⁸ RFP-labeled Salmonella typhimurium was injected in the GFP-labeled PC-3 human prostate tumor growing in a nude mouse (FIG. 10A) and imaged using techniques similar to Example 1 immediately after injection (FIG. 10B) Growth of RFP-labeled Salmonella typhimurium in the GFP-labeled PC-3 human prostate tumor was seen one day after injection (FIG. 10C) showing RFP-labeled Salmonella growth around the tumor and reduction of tumor size.

### EXAMPLE 8

### Targeting in Prostate Tumor Using Whole-Body Imaging

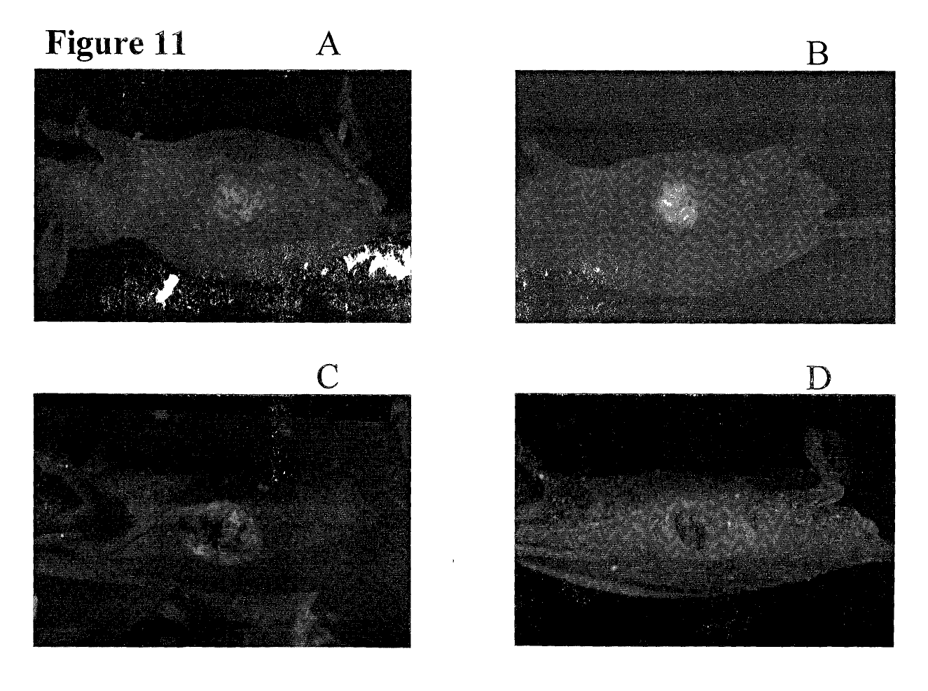

A solution containing 2×10⁸ RFP-labeled Salmonella typhimurium was injected in the GFP-labeled PC-3 human prostate tumor growing in a nude mouse (FIG. 11A) and imaged using techniques similar to Example 1 immediately after injection (FIG. 11B). RFP-labeled Salmonella was detected as growing in the GFP-labeled PC-3 human prostate tumor one day after injection (FIG. 11C) and continuing to grow in the tumor four days after injection (FIG. 11D) while reduction of tumor size is shown.

### EXAMPLE 9

### Targeting in Prostate Tumor Using Whole-Body Imaging

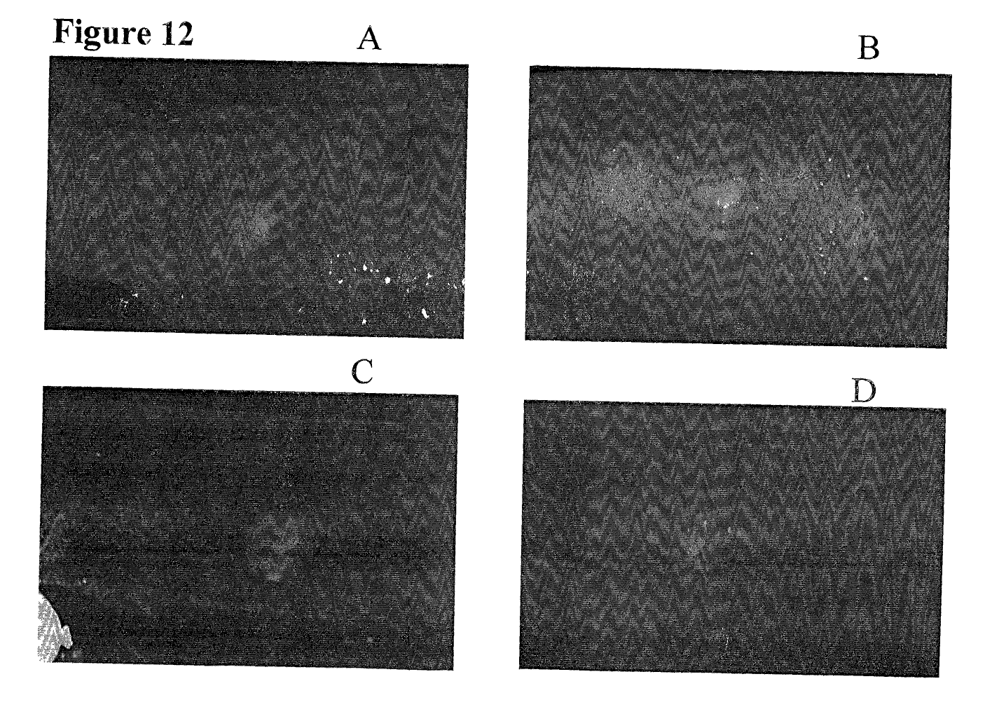

A solution containing 2×10⁸ RFP-labeled Salmonella typhimurium was injected in the GFP-labeled PC-3 human prostate tumor growing in a nude mouse (FIG. 12A) and imaged using techniques similar to Example 1 immediately after injection (FIG. 12B). RFP-labeled Salmonella is seen growing in the GFP-labeled PC-3 human prostate tumor one day after injection (FIG. 12C) and four days after injection (FIG. 12D) showing visible reduction in tumor size.

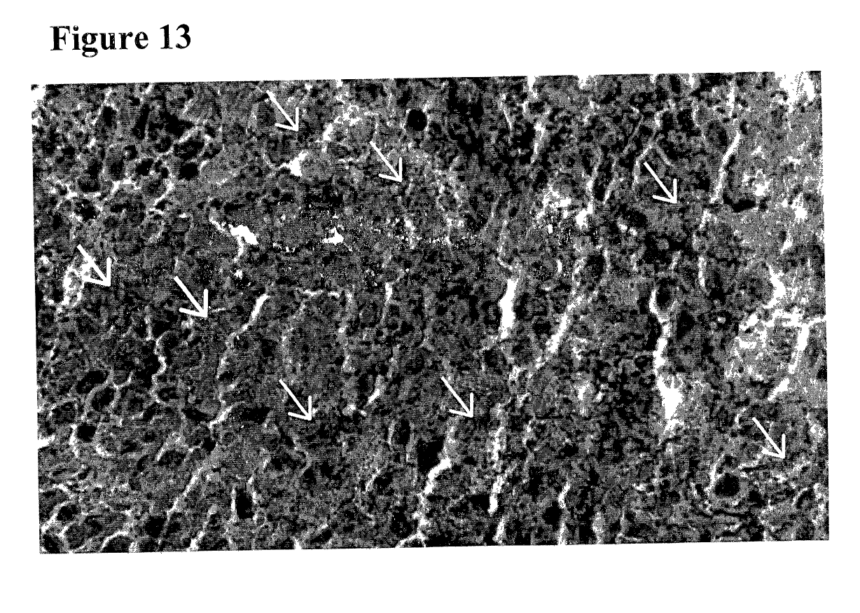

Histological studies were performed on the RFP-labeled Salmonella growing in the GFP-labeled PC-3 human prostate tumor four days after injection (FIG. 12D) by fixing the tumor tissue with 10% buffered formaline and processed for paraffin section and HE staining by standard method. The RFP-labeled Salmonella (the small blue dots as pointed by the white arrows in FIG. 13, Magnification 400X) were progressively growing in the PC-3 tumor tissue and targeting the tumor cells.

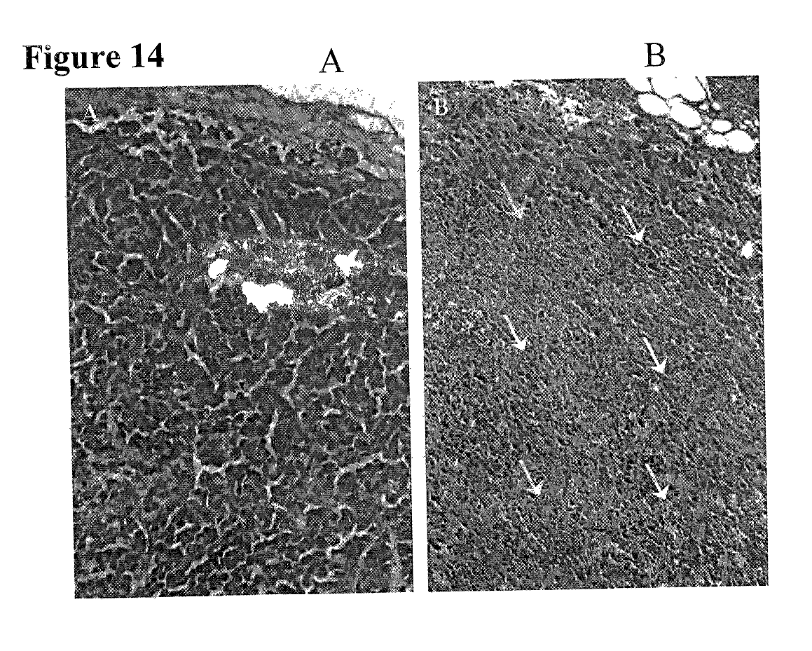

Histological studies (HE staining. Magnification 200X) also compared an untreated control showing a well-maintained PC-3 human prostate tumor structure growing in a nude mouse (FIG. 14A) with PC-3 human prostate tumor growing in a nude mouse treated with RFP-labeled Salmonella four days after injection (FIG. 14B). The majority of tumor tissue has been destroyed, and the extensive necrosis (arrows in FIG. 14B) in the tumor is shown.

1. A method to monitor the progression of infection in a vertebrate subject which method comprises
observing the presence, absence or intensity of fluorescence at various locations in said subject as a function of time, wherein said vertebrate subject has been treated with an infective agent that expresses a fluorescent protein.

2. The method of claim 1, wherein said observing is by endoscopy or fluorescent optical tumor imaging in the intact subject.

3. The method of claim 1, wherein the fluorescent protein has green fluorescence or red fluorescence.

4. The method of claim 1, wherein the infective agent is a bacterium, protozoan or virus.

5. The method of claim 1, wherein the infective agent is a eukaryotic single-celled organism.

6. The method of claim 1, wherein the subject is a mammal.

7. The method of claim 6, wherein the subject is a mouse, rat or rabbit.

8. The method of claim 1, wherein the subject is immunocompromised.

9. A method to evaluate a candidate protocol or drug for the inhibition of infection which method comprises
administering said protocol or drug to a vertebrate subject which has been treated with an infective agent that expresses a fluorescent protein and monitoring the progression of infection over time by observing the presence, absence or intensity of the fluorescence at various locations in said treated subject;
monitoring the progression of infection over time in a control subject which has been similarly treated with an infective agent that expresses a fluorescent protein; and
comparing the progression of infection in said treated subject with the progression of infection in said control subject;
whereby a diminution of the progression of infection in said treated subject as compared to said control subject identifies the protocol or drug as effective in inhibiting infection.

10. The method of claim 9, wherein said monitoring is by endoscopy or fluorescent optical tumor imagining in the intact subject.

11. The method of claim 9, wherein the infective agent is a bacterium, protozoan or virus.

12. The method of claim 9, wherein the infective agent is a eukaryotic single-celled organism.

13. The method of claim 9, wherein the subject is a mammal.

14. The method of claim 13, wherein the subject is a mouse, rat or rabbit.

15. The method of claim 9, wherein the subject is immunocompromised.

16. A method to identify genes associated with infection, which method comprises observing the presence, absence or intensity of fluorescence in said subject as a function of time and location in said subject, wherein said subject has been infected with an infective agent with an altered genome, wherein the alteration in the genome comprises replacement of a nucleotide sequence whose function is to be determined with a nucleotide sequence encoding a fluorescent protein.

17. The method of claim 16, wherein the infective agent is a bacterium, protozoan or virus.

18. The method of claim 16, wherein the infective agent is a eukaryotic single-celled organism.

19. The method of claim 16, wherein the subject is a mammal.

20. The method of claim 19, wherein the subject is a mouse, rat or rabbit.

21. The method of claim 16, wherein the subject is immunocompromised.

22. The method of claim 16, wherein said observing is by endoscopy or fluorescent optical tumor imaging in the intact subject.

23. A method of targeting tumors using a therapeutic infective agent in a vertebrate subject comprising
administering an infective agent that expresses a fluorescent protein to said vertebrate subject containing a tumor; and
observing the presence, absence or intensity of fluorescence in said subject as a function of time.

24. The method of claim 23, wherein the therapeutic infective agent delivers a therapeutic product to the tumor.

25. The method of claim 23, wherein the tumor exhibits fluorescence of a color other than the color of the infective agent.

26. The method of claim 24, wherein the therapeutic product exhibits fluorescence of a color different than the color of the infective agent and the tumor.

27. The method of claim 23, wherein the infective agent is a bacterium, a protozoan or a virus.

28. The method of claim 23, wherein the infective agent is a eukaryotic single-celled organism.

29. The method of claim 23, wherein the subject is a mammal.

30. The method of claim 29, wherein the subject is a mouse, rat or rabbit.

31. The method of claim 22, wherein the subject is immunocompromised.

32. The method of claim 22, wherein said observing is by endoscopy or by fluorescent optical tumor imaging in the intact subject.

33. The method of claim 23, wherein tumor necrosis is induced.

34. A tumor targeting infective agent comprising an infective agent that expresses a fluorescent protein, where the infective agent is capable of preferentially targeting the tumor in an intact, living mammal in comparison to normal cells.

35. A tumor targeting infective agent as in claim 34, wherein the infective agent contains or secretes a therapeutic molecule or contains a gene of interest.
